(19) United States
(12) Patent Application Publication
(10) Pub. No.: US 2014/0288387 A1
(43) Pub. Date: Sep. 25, 2014

(54) INTEGRATED PATIENT HEALTH TRACKING SYSTEM

(71) Applicant: David Duplay, New York City, NY (US)

(72) Inventor: David Duplay, New York City, NY (US)

(21) Appl. No.: 14/221,855

(22) Filed: Mar. 21, 2014

Related U.S. Application Data
(60) Provisional application No. 61/804,272, filed on Mar. 22, 2013.

(51) Int. Cl. A61B 5/00; A61B 5/0205

(52) U.S. Cl. CPC A61B 5/7275; A61B 5/0205
USPC 600/301

(57) ABSTRACT

System in support of managing patient support for patients suffering from various ailments includes a portable health monitoring or tracking device, a computer, a processor, a memory and a display. A user interface receives patient health-related and support information and enables navigation. Modules execute to configure the processor in certain ways, including a patient profile module which defines a custom patient profile which includes personal factors that relate to at least one ailment, a patient similarity module which processes and organizes the patient profiles within ailment classes by weighing the personal factors and calculating a degree of a similarity between the patient profile and other patient profiles within the same ailment class. A first content module provides an output to users in which content is displayed in order of a similarity between the patient profile and the other patient profiles in the same ailment class. Further modules can be provided to track content-viewing tendencies and assess users' ailment concerns.

### CROSS REFERENCE TO RELATED APPLICATIONS

This application claims the benefit under 35 U.S.C. 119(e) of U.S. Provisional Patent Application Ser. No. 61/804,272, filed on Mar. 22, 2013, entitled “h360Tracker,” which is hereby incorporated by reference as if set forth in its entirety herein.

### FIELD OF THE INVENTION

The present invention relates to the field of social networking. More particularly, the present invention relates to a system including a health tracking device integrated with a social network for patients of various ailments. As such, the present invention provides an on-line platform where patients can come together and receive content that are most helpful to their medical situation.

### BACKGROUND

The continued growth of the Internet and social networking has greatly expanded the capabilities and ease of providing and consuming information. It has become common to share, search and rely on a variety of information over the Internet. Patients often have the information needs to understand the particular aspects of their situation, thus are a special group among all the Internet users. In addition, to an individual patient, information and support from Internet users that suffer from similar ailments are likely most valuable. As such, medical information, relating to ailment, treatment, recovery and other associated concerns, is of one kind which value is individual-specific. However, the existing medical information available over the Internet is unorganized, and difficult to be searched and relied on by an individual patient.

Accordingly, it is desirable to be able to build a social network to maximize the information value by grouping a patient with other patients of similar conditions. Besides allowing patients to connect with each other and share medical information, it is also desirable to be able to proactively supply users with customized information relating to the ailment they suffer from. This way, users can be benefitted from the support and information received from the network in the process of recovery.

In addition, it would be very beneficial for patients to have a system that allows for both the monitoring and sharing of health-related information via a web-based social networking interface.

### SUMMARY

The description provides a system that comprises a portable device for monitoring and tracking of patient health-related information, which is integrated with a computer-based system for managing a patient support and information network for patients suffering from various ailments. The system includes a device for monitoring and/or tracking health-related information, a computer having a processor, a memory, a user interface, and a display. In certain embodiments, the device monitors or tracks (manually or automatically) health-related information such as, e.g., physical activity, sleep, diet, caloric intake, metabolic rate, heart rate, pulse, mood, medications, compliance with dosage regimen, response to medication, side effects to medication, or the like. Patients can upload the data from the device via a wired connection (e.g., USB) or wireless connection, e.g., Bluetooth® or Wi-Fi™, to a social networking user interface such as is described herein.

In certain aspects, the user interface is configured to receive input, e.g., from a portable device that monitors or tracks health-related information, from a user and enable navigation through the network. In certain embodiments, the user input is health-related information from a monitoring or tracking device, which is storable in the memory and which is displayable on command to the user through the user interface. In certain other embodiments, the user input includes patient support information which is storable in the memory and which is displayable on command to the user through the user interface. In a preferred embodiment, a patient profile module executes in the processor and configures the processor to define a custom patient profile using the user interface. The patient profile can be stored in the memory, and includes personal factors that relate to at least one ailment from which the user suffers. In additional embodiments, a patient similarity module executes in the processor and configures the processor to process the stored patient profiles based on the at least one ailment. The patient similarity module further configures the processor to organize the patient profile within each ailment class by weighing the personal factors in the patient profile and by calculating a degree of a similarity between the patient profile and other patient profiles within the same ailment class based on the personal factors of the users. In certain additional embodiments, a first content module executes in the processor and configures the processor to output the information inputted by the other users as content and to display the content on the display in an order of degree of similarity between patient profile and the other patient profiles in the same ailment class.

In accordance with a further aspect of the invention, a computer-based system as described above can further include an ailment concern module which executes in the processor and which configures the processor to track a user's content-viewing tendency in the network and to assess the user's ailment concerns reflected by the content-viewing tendency. Such a system in accordance with this further aspect can include (a) a second content module which executes in the processor and which configures the processor to match the content stored in the memory with the user's ailment concerns assessed by the ailment concern module and output content that provides support information of such concerns, and (b) a content organization module that allows the user to rate the content supplied by the first and second content modules.

The preceding general areas of utility are given by way of example only and are not intended to be limiting on the scope of the present disclosure and appended claims. Additional objects, features and advantages associated with the compositions, methods, and processes of the present invention will be appreciated by one of ordinary skill in the art in light of the instant claims, description, and examples. For example, the various aspects and embodiments of the invention may be utilized in numerous combinations, all of which are expressly contemplated by the present description. These additional advantages objects and embodiments are expressly included within the scope of the present invention. The publications and other materials used herein to illuminate the background of the invention, and in particular cases, to provide additional details respecting the practice, are incorporated by reference.

### BRIEF DESCRIPTION OF THE DRAWING FIGURES

The accompanying drawings, which are incorporated into and form a part of the specification, illustrate several embodiments of the present invention and, together with the description, serve to explain the principles of the invention. The drawings are only for the purpose of illustrating an embodiment of the invention and are not to be construed as limiting the invention. Further objects, features and advantages of the invention will become apparent from the following detailed description taken in conjunction with the accompanying figures showing illustrative embodiments of the invention, in which:

FIG. 1 is an exemplary computing system environment that can be used in an embodiment of the invention.

FIG. 2 is an example of the user interface that can be used in an embodiment of the invention.

FIG. 3 illustrates a different part of the user interface than shown in FIG. 2, in which content is displayed and navigation controls are provided, as can be used in an embodiment of the invention.

FIG. 4 illustrates a different part of the user interface than shown in FIGS. 2 and 3, in which logged-in users manage their account through the user interface, as can be used in an embodiment of the invention.

FIG. 5 is another part of the user interface of FIG. 4, as can be used in an embodiment of the invention, now showing data capture that is used in connection with the analysis and processing of content described more fully hereinbelow.

### DESCRIPTION OF CERTAIN EMBODIMENTS OF THE INVENTION

The following is a detailed description provided to aid those skilled in the art in practicing the present invention. Those of ordinary skill in the art may make modifications and variations in the embodiments described herein without departing from the spirit or scope of the present disclosure. All publications, patent applications, patents, figures and other references mentioned herein are expressly incorporated by reference in their entirety.

By way of overview and introduction, the invention described herein concerns a computer-based system for managing a patient support and information network. The system is directed to assisting users suffering ailments to share and connect with patients of similar conditions. The system is further directed to proactively supply information to a user that is relevant to his ailment or concern. Through the system, users are able to better manage their conditions and foresee other aspects of recovery.

Unless otherwise defined, all technical and scientific terms used herein have the same meaning as commonly understood by one of ordinary skill in the art to which this invention belongs. The terminology used in the description is for describing particular embodiments only and is not intended to be limiting of the invention.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

The following terms are used to describe the present invention. In instances where a term is not specifically defined herein, that term is given an art-recognized meaning by those of ordinary skill applying that term in context to its use in describing the present invention.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

In one aspect, the description provides a system that comprises a portable device for monitoring and tracking of patient health-related information, which is integrated with a computer-based system for managing a patient support and information network for patients suffering from various ailments. The system includes a portable device for monitoring and/or tracking health-related information, a computer having a processor, memory, a user interface, and a display. In certain embodiments, the system includes a database of stored user health information. In certain embodiments, the portable device monitors or tracks (manually or automatically) health-related information such as, e.g., physical activity, sleep, diet, caloric intake, metabolic rate, heart rate, pulse, mood, medications, compliance with dosage regimen, response to medication, side effects to medication, or the like. Patients can upload the data from the device via a wired connection (e.g., USB) or wireless connection, e.g., Bluetooth® or Wi-Fi™, to a social networking user interface such as is described herein. Currently available devices that can be used in conjunction with the systems as described herein, include the Nike Fuel Band®, Jawbone Up®, and FitBit®.

The monitoring or tracking device is an innovative tool to capture health information about a user. In certain embodiments, the information is monitored or tracked, and recorded automatically by a device worn or carried, self-reported by a user by inputting data into, e.g., a mobile device, which interfaces with both the worn device, or both; and a web-based platform as described herein. In certain embodiments, the tracked and reported information is stored in a user's “My Account” section of the web-based interface, which can be marked private or shared with the entire community or selected members. Once this information is uploaded into the web-based platform, a user can compare themselves to other users who are just like them. In certain embodiments, the web-based interface includes a “tracker support group”; i.e., users that are using monitoring or tracking devices to monitor and track health and life information. In additional embodiments, the users can communicate with each other in the group discussion or mark the conversation as private, and select only those members invited into the discussion. In a preferred embodiment, users can make some, none or all information monitored or tracked private or visible to the web-based platform community of users.

In certain embodiments, the portable health monitoring or tracking device may be configured to monitor or track health-related information, including, e.g., physical activity, sleep, diet, calorie intake, metabolic rate, heart rate, pulse, mood, medications, compliance with dosage regimen, response to medication, side effects to medication, or the like.

In addition embodiments, the device is configured to monitor or track at least one of the following:

(i) Sleep habits. In certain embodiments, the portable health monitoring or tracking device is configured to monitor or track, e.g., hours slept, light vs. deep sleep, waking moments or a combination thereof. For example, the device can be configured to automatically monitor and upload sleep activity information or the information can be manually entered into the device, a mobile phone, or web-based interface by the user. Users can then compare their sleep habits to other users, including the entire online community or their specific groups.

(ii) Physical activity: In certain additional embodiments, the portable health monitoring or tracking device is configured to monitor or track movements, including distance, calories burned, activity duration and/or intensity or a combination thereof For example, the device can be configured to automatically monitor and upload information about a user's physical activity or the information can be manually entered into the device, a mobile phone, or web-based interface by the user. Users can then compare their physical activity to other users, including the entire online community or their specific groups.

(iii) Nourishment: In certain embodiments, the portable health monitoring or tracking device is configured to monitor at least one of food, drink, calories, or combination thereof consumed during the day. In certain embodiments, the device includes a scanner/QR reader or a mobile device can be used for convenient entry of information and uploading into the database. Users can then compare their diet to other users, including the entire online community or their specific groups.

iv) Temperament: In certain embodiments, the portable health monitoring or tracking device is configured to monitor or track user mood or disposition over a desired time flame, e,g., hourly, daily, weekly, monthly, etc. Tracking such data can allow users to discover connections and patterns that affect how they feel, including correlations with social interaction, activity level, diet, and/or medication. In certain embodiments, data regarding a user's mood is manually entered to a tracking device, mobile phone or web-based interface. Users can then compare their mood to other users, including the entire online community or their specific groups.

(v) Medication: In certain embodiments, the portable health monitoring or tracking device is configured to monitor or track at least one of user medication type, dosage, regimen, reaction or combination thereof over a desired time frame, e.g., hourly, daily, weekly, monthly, yearly, etc. Tracking such data can allow users to discover connections and patterns relating to one or more medications. In certain embodiments, data regarding a user's medication type, dosage, regimen, reaction or combination thereof is manually entered to a tracking device, mobile phone or web-based interface. Users can then compare their data to other users, including the entire online community or their specific groups.

(vi) System engagement: in certain embodiments, the portable health monitoring or tracking device is configured to monitor or track use of the system by a user over a desired time frame, e.g., hourly, daily, weekly, monthly, yearly, etc. In certain embodiments, data can be tracked, e.g., how many times a user logs on to the system, how many stories are reviewed, how many support/sponsor pages were visited or a combination thereof. In certain embodiments, data regarding a user's engagement with the system is manually entered to a tracking device, mobile phone or web-based interface. Users can then compare their data to other users, including the entire online community or their specific groups.

In a preferred embodiment, the description provides a system for managing a patient health-related information network comprising a portable health monitoring or tracking device configured to monitor or track at least one of patient physical activity, sleep, diet, temperament, caloric intake, heart rate, pulse, mood, medication, dosage regimen, response to medication or combination thereof; a computer system having a processor and a memory, wherein the computer system is configured to receive input monitored or tracked by the portable health monitoring or tracking device automatically or manually; a user interface configured receive input from a portable health monitoring or tracking device and/or a user, which enables navigation through the network, the input including patient physical activity, sleep, diet, temperament, caloric intake, heart rate, pulse, mood, medication, dosage regimen, response to medication, patient support information or a combination thereof, storable in the memory and displayable on command to the user; a patient profile module executing in the processor and configuring the processor to define a custom patient profile using the user interface, the patient profile being stored in the memory, wherein the patient profile includes personal factors that relate to at least one ailment from which the user suffers; a patient similarity module executing in the processor and configuring the processor to process the stored patient profiles based on the at least one ailment, the patient similarity module further configuring the processor to organize the patient profile within each ailment class by weighing the personal factors in the patient profile and calculating a degree of a similarity between the patient profile and other patient profiles within the same ailment class based on the personal factors of the users; a first content module executing in the processor and configuring the processor to output the inormation inputted by the other users as content and display the content in an order of degree of similarity between patient profile and the other patient profiles in the same ailment class; and a display for displaying the output content.

It is to be appreciated that the embodiments of this invention as discussed herein may be incorporated as a software algorithm, application, program or code residing in hardware, firmware and/or on a computer useable medium (including software modules and browser plug-ins) that can be executed in a processor of a computer system to configure the processor to perform the functions described below. Such a computer system typically includes memory storage configured to provide output from execution of the computer algorithm, code, or program. The processor may be configured to execute one or more modules.

The computer system preferably includes a processor (such as a digital data processor), a memory, and a control circuit. The memory stores data and instructions suitable for controlling the operation of processor. An implementation of memory can include, for example, a random access memory (RAM), a hard drive and a read only memory (ROM), or any of these components.

One of the components stored in memory is a program. Program includes instructions for controlling processor to perform particular tasks or implement particular abstract data types. Program may be implemented as a plurality of modules that operate in cooperation with one another. Program is contemplated as representing a software embodiment, or a component or module thereof, of the method described herein.

In embodiments of the present invention, portions of the program including selected modules or code thereof can execute entirely without user input or other commands based on programmatic or automated access to a data signal flow through other systems that may or may not require a user interface for other reasons.

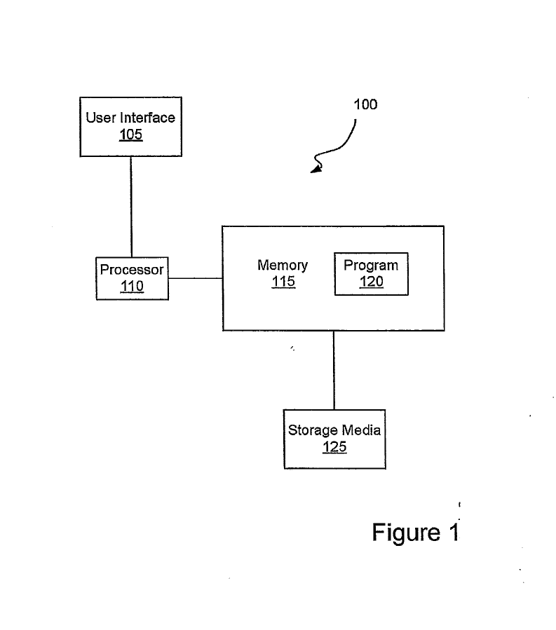

With the exemplary computing system environment of FIG. 1 being generally shown and discussed above, the system of the invention in accordance with illustrated embodiments are also discussed herein.

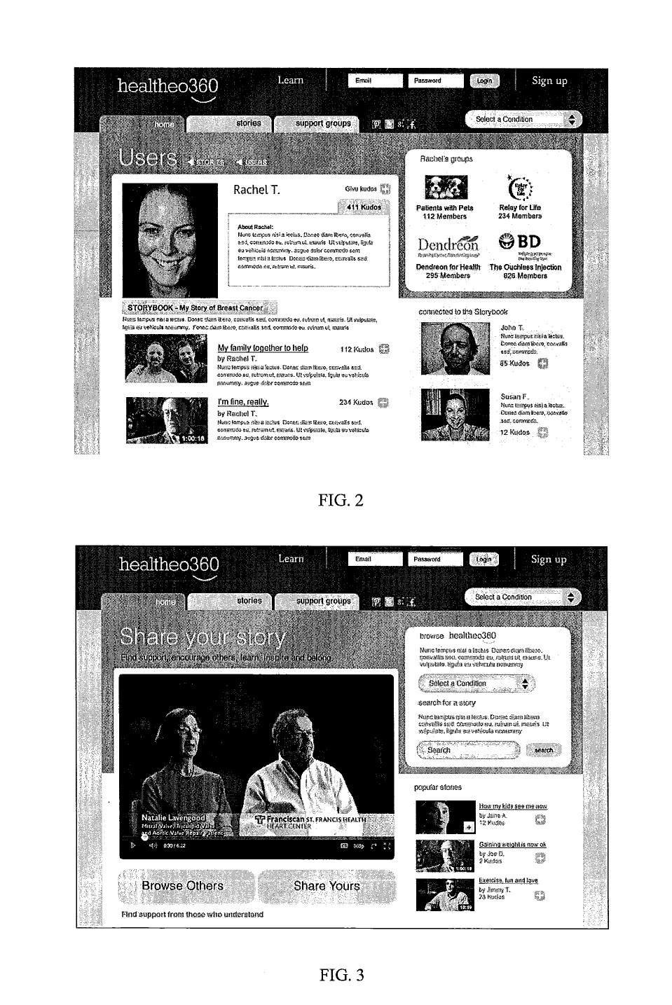

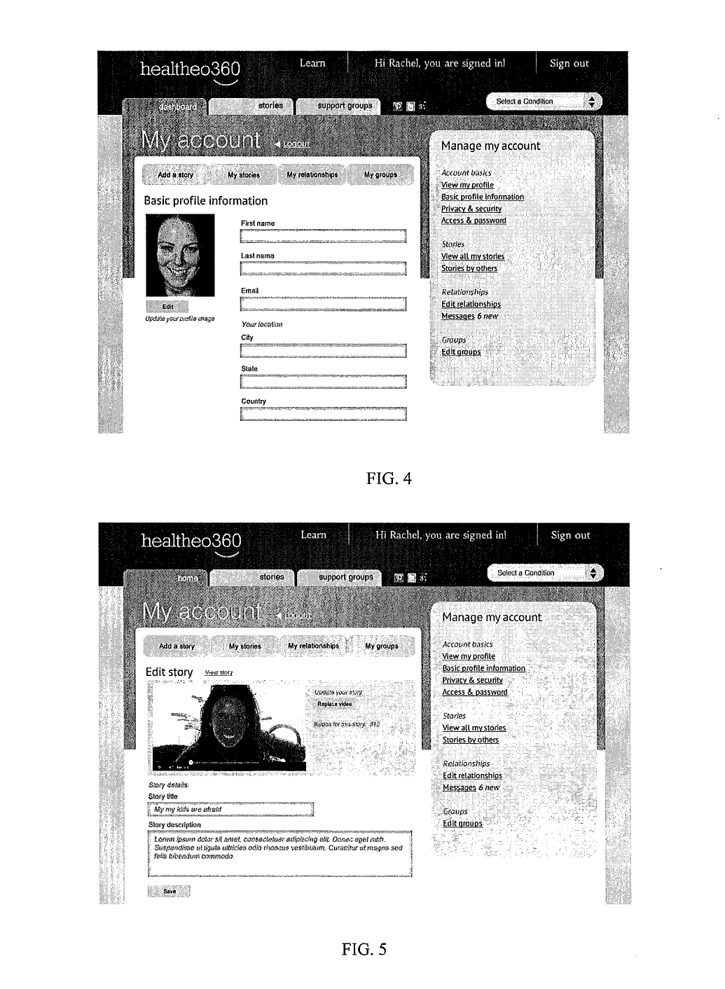

In one embodiment, the user interface can comprise a plurality of interactive pages that are delivered on demand to users seated at devices connectable to various computer networks such as the Internet. Users interact with the pages using devices having a processor, memory, a network interface card (NIC), and navigation software such as a web browser program. Preferably, the pages that comprise the user interface are compatible with one or more conventional web browsers. FIGS. 2 through 5 illustrate examples of pages in accordance with the illustrated embodiment of the invention that can be rendered in conventional browsers used by any number of users. The users input information and command selections to the processor of their respective devices and such information and commands are provided to the system of the present invention (e.g., a server which is a computer having at least one processor and memory or access to memory) for processing and delivery of further information.

A set of modules cooperate with one another to provide the information presented through the interface. It is possible to envision to all of the disclosed features and capabilities of the present invention as software modules. These modules are stored as computer code in memory and are executed by the processor. The modules described hereunder include a patient profile module, a patient similarity module, a first content module, an ailment concern module, a second content module and a content organization module that are executed by processor.

The system also includes an output device such as a display, a touch screen or a data terminal. In the case of a touch screen, the input and output functions are provided by the same structure.

FIG. 2 is an example of the user interface. In one aspect, the user interface is configured to provide access to various information input and editing functions enabled for a user. In another words, a user of the system can input information through the user interface. The information a user can input include information identifying the user, content submitted by the user, and other information.

The content submitted by the user can take various forms, including text, images and videos, or any combination thereof. The exemplary user interface also allows the user to upload videos and images. The content submitted by the user through the user interface is transmitted by the processor and stored in the memory in association with that individual user.

An exemplary user interface is similar to Microsoft Word where a user can edit, delete, copy and paste text to create content. In several embodiments, a user can input information regarding ailment, treatment, recovery, or other associated concerns. Examples of such content include, but are not limited to:

Feelings after diagnosis—emotional (how to discuss the illness with family, friends, and work colleagues), what can be expected in the early days post diagnosis, what have others done as they began searching for a treatment plan, how to maintain a positive outlook

Disease progression—what might happen as the disease progresses and what the patient might expect

Tips and suggestions on how to live with the condition

Tips and suggestions on overcoming fears

Lifestyle change requirements and how to make the best of each situation

Support group information for patients and caregivers

Local directories of support groups and other qualified facilities or groups

Clinical trial information—when, where and qualifications

The user is presented with editing tools in the user interface. A user can modify previously inputted content through the user interface by the editing tools executed by the processor. The editing tools enable a user to view a list of the content associated with that user. Upon the user's command on the editing tools, the information to be edited is retrieved from the memory and displayed at the user interface. The user may preview the changes, save and/or discard the changes. The edited content is stored in the memory in association with that individual user.

In one aspect, a user inputs his personal factors through the user interface to create a patient profile. The creation and management of information identifying a user is configured by the patient profile module as described below. In one aspect, a user can determine whether the particular inputted information is accessible by other users.

FIG. 3 illustrates an exemplary user interface for displaying and navigating content through the user interface. In one aspect, each content module described below is selectable by a user to access a preview of the content that is displayed within content module. Additionally, the preview may further provide access to the additional content, which may open or launch a new window or application to access the additional content.

As described, the content modules may be associated with different types of content such as text, images and videos, combinations thereof, and any other content suitably displayed on the user interface.

The patient profile module is configured to manage patient profiles associated with users of the system. The patient profile module executes in the processor and configures the processor to define a patient profile. The patient profile module enables a user to input and modify the information in the user profile through the user interface.

A user profile contains basic individual information of a user such as: first name, last name, email address, user name, the creation of a password, geography location (city and state), and a short description about their ailments. The information contained in a user profile is also modifiable through the user interface.

Preferably, the ailments information inputted by a use includes the ailment(s) the individual suffers, as well as the associated degree of severity, treatment plans, and degree of recovery.

In one aspect, a user can select the class of ailment that his conditions fall under. Applicable aliment classes include but are not limited to; Asthma, Blood Cancer, Breast Cancer, Cardiovascular Disease, Colon Cancer, CORD, Diabetes Type 1, Diabetes Type 2, High Cholesterol, High Triglycerides, Hip Replacement, Hypertension, Knee Replacement, Lou Gehrig's Disease, Multiple Myeloma, Osteo Arthritis, Pain Management, Parkinson's Disease, Prostate Cancer, Rheumatoid Arthritis, Skin Cancer, Testicular Cancer.

A patient similarity module is configured to assemble patient profiles, and to further assess similarities of users of the system based on the information contained in the patient profiles.

In one embodiment, the patient similarity module executes in the processor and configures the processor to process stored user profiles in the memory. Specifically, the module configures the processor to organize patient profiles on the network based on the ailment class indicated in their profile. As such, users who indicate that they suffer a same class of ailment are grouped.

Preferably, if a user selected more than one ailment class in the patient profile, the patient similarity module configures the processor to process the patient profile each time his ailment class is grouped.

The patient similarity module further configures the processor to organize the patient profiles within each ailment class by weighing the personal factors of the users. Such personal factors include age, sex, geography, degree of severity and degree of recovery. According to the weight assigned to each personal factor, the patient similarity module calculates a degree of similarity between a particular patient profile and other patient profiles within the same ailment. In a preferred embodiment, the personal factors of degree of severity and/or degree of recovery must be weighed in to calculate the degree of similarity among the users of the same ailment class. The calculated degree of similarity is stored in the memory in association with the individual user.

A first content module is configured to supply content to a user based on the associated degree of similarity for that individual user in his ailment class. Specifically, a first content module retrieves the stored content submitted by the other users in the same ailment class from the memory. The first content module further organizes the retrieved content in the order of the degree of similarity between a particular patient profile and the other patient profiles in the same ailment class.

For example, in the ailment class of asthma, there are four (4) users. User A, User B and User C all suffer from stage 1 asthma. User D nevertheless suffers from stage 4 asthma. Under the calculation of the patient similarity module for User A, User B and User C would score a higher degree of similarity to User A than User D. Moreover, User B will score an even higher degree of similarity than User C if User B is of the same age group as User A, but User C isn't. Thus, when the first content module is configured to output content from similar users for User A in his ailment class, the content created by User B will be displayed on the highest hierarchy level, followed by content created by User C and User D.

The content assembled by the first content module for individual user is accessible in the user interface. Specifically, the content is displayable to the user on the display device upon selection in the user interface.

The ailment concern module is configured to track a user's content-viewing tendency in the network and to assess the user's ailment concerns. In one aspect, the ailment concern module configures the processor to track indicators that reflect the content-viewing tendency at least in conjunction with the ailment from such the user suffers.

Indicators that are relevant and to be tracked by the ailment concern module include but are not limited to the dwell time on a webpage and the frequency of clicks on particular hyperlinks.

Preferably, a set of ailment concerns are pre-determined and stored in the ailment concern module for each ailment class grouped in the system. After collecting sufficient amount of data regarding the content-viewing tendency for an individual user, the ailment concern module determines the score of each ailment concern for the user. The module further configures the processor to generate a hierarchy of aliment concerns for that user based on the score of the ailment concern for the user. The hierarchy of aliment concerns is stored in the memory in association with the individual user.

A second content module is configured to cause more relevant information to be displayed to the user. The second content module executes the processor to match the content in memory with the user's ailment concerns determined by the ailment concern module and outputs such content. Preferably, the matching content is outputted in the hierarchy of ailment concerns determined for that individual user and stored in the ailment concern module.

The content matched by and stored in the second content module for an individual user is accessible in the user interface. Specifically, the content is displayable to the user on the display device upon selection in the user interface.

For example, for users who are grouped in the ailment class of breast cancers, the ailment concern module executes the processor to track the content-viewing tendency indicators and determine if a user has the following specific ailment concerns associated with breast cancer:

Newly Diagnosed

Daily Care Routines

Diet & Exercise

Communicating with Family & Friends

Husbands' Network

Chemotherapy

Treatment Regimens

Mastectomy

Therapy & Support Groups

After the ailment concern module records a high clicking record and/or a long dwell time for content related to Daily Care Routines for an individual user, the second content module searches and matches the content related to Daily Care Routines stored in the memory, and outputs the matching content for that user.

By way of further example, for users who are grouped in the ailment class of diabetes, the ailment concern module configures the processor to track the content-viewing tendency indicators and determine if a user has the following ailment concerns associated with diabetes.

Newly Diagnosed

Daily Care Routines

Diet & Exercise

Healthy Habits

Communicating with Family & Friends

Blood Sugar Testing

Treatment Regimens

Therapy & Support Groups

After recording a user has a high clicking rate and/or a long dwell time for content related to several ailment concerns, including Daily Care Routines, Diet & Exercise and Blood Sugar Testing, the ailment concern module generates a hierarchy of aliment concerns according to the recorded content-viewing tendency for each concern. The second content module further configures the processor to search and match the content related to Daily Care Routines, Diet & Exercise and Blood Sugar Testing in the memory, and outputs the matching content in the hierarchy of ailment concerns determined for that user.

Through the user interface, a user receives ailment-related content from various sources, including but are not limited to the content created by other patients on the network and supplied by the first and the second content module. A display module is configured to display the content upon the command of the user,

A content organization module is configured to organize the content according to a user's preference. For example, a user can rate the content supplied by the first and second content modules in the user interface. Specifically, the user can change the hierarchy of the content by moving up and down of a specific piece of content. The user can also remove a specific piece of content from his user interface. After the content removal is effectuated, the content organization module configures the processor to match the content stored in the memory with the content that is removed by the user. Accordingly, whenever the first and second content modules are configured to generate content for that user, the content organization module filters out the content that is matched with the removed content. As such, the user may no longer view the content which he is not preferred.

The present invention is described more fully with reference to the accompanying drawings, in which one or more illustrated embodiments of the invention are shown. The invention is not limited in any way to the illustrated embodiments as the illustrated embodiments described herein are merely exemplary of the invention, which can be embodied in various forms, as appreciated by one skilled in the art. Therefore, it is to be understood that any structural and functional details disclosed herein are not to be interpreted as limiting the invention, but rather are provided as a representative embodiment for teaching one skilled in the art one or more ways to implement the invention. Furthermore, the terms and phrases used herein are not intended to be limiting, but rather are to provide an understandable description of the invention.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

The contents of all references, patents, pending patent applications and published patents, cited throughout this application are hereby expressly incorporated by reference.

Those skilled in the art will recognize, or be able to ascertain using no more than routine experimentation, many equivalents to the specific embodiments of the invention described herein. Such equivalents are intended to be encompassed by the following claims. It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

What is claimed is:

1. A system for managing a patient health-related information network comprising:
a portable health monitoring or tracking device configured to monitor or track at least one of patient physical activity, sleep, diet, temperament, caloric intake, heart rate, pulse, mood, medication, dosage regimen, response to medication or combination thereof;
a computer system having a processor and a memory, wherein the computer system is configured to receive input monitored or tracked by the portable health monitoring or tracking device automatically or manually;
a user interface configured receive input from a portable health monitoring or tracking device and/or a user, which enables navigation through the network, the input including patient physical activity, sleep, diet, temperament, caloric intake, heart rate, pulse, mood, medication, dosage regimen, response to medication, patient support information or a combination thereof, storable in the memory and displayable on command to the user;
a patient profile module executing in the processor and configuring the processor to define a custom patient profile using the user interface, the patient profile being stored in the memory, wherein the patient profile includes personal factors that relate to at least one ailment from which the user suffers;
a patient similarity module executing in the processor and configuring the processor to process the stored patient profiles based on the at least one ailment, the patient similarity module further configuring the processor to organize the patient profile within each ailment class by weighing the personal factors in the patient profile and calculating a degree of a similarity between the patient profile and other patient profiles within the same ailment class based on the personal factors of the users;
a first content module executing in the processor and configuring the processor to output the information inputted by the other users as content and display the content in an order of degree of similarity between patient profile and the other patient profiles in the same ailment class; and
a display for displaying the output content

2. The system of claim 1 further comprising:
an ailment concern module executing in the processor and configuring the processor to track a user's content-viewing tendency in the network and assess the user's ailment concerns reflected by the content-viewing tendency;
a second content module executing in the processor and configuring the processor to match the content stored in the memory with the user's ailment concerns assessed by the ailment concern module and output content that provides support information of such concerns; and
a content organization module that allows the user to rate the content supplied by the first and second content modules.

3. The system of claim 2, wherein the personal factors include at least two of age, sex, geography, degree of severity and degree of recovery.

4. The system of claim 3, wherein the personal factors at least includes degree of severity and degree of recovery.

5. The system of claim 2, wherein the viewing patterns include a dwell time on a webpage.

6. The system of claim 2, wherein the viewing patterns include a frequency of clicks on particular hyperlinks.

7. The system of claim 2, wherein the content organization module is further configured to permit the user to remove the content supplied by the first and second content module.

8. The system of claim 5, wherein the first and second content module is further configured to filter information that is associated with the content that has been removed by the user.

9. The system of claim 1, wherein the personal factors include at least two of age, sex, geography, degree of severity and degree of recovery.

10. The system of claim 9, wherein the personal factors at least includes degree of severity and degree of recovery.
